SEQUENCE LISTING (13 sequences)

SEQ ID NO: 1 - protein, 32 aa, synthetic construct
AIIFIASANHKVTPTTAIIQDATSQIKNTPPS

SEQ ID NO: 2 - protein, 91 aa, synthetic construct
MIISTSLIIAAIIFIASANHKVTPTTAIIQDATSQIKNTPPSKPNNDFHFEVFNFVPCSICSNNPTCWAICKRIPNKKPGKKTTTKPTKKP

SEQ ID NO: 3 - protein, 138 aa, synthetic construct
MSKNKDQRTAKTLERTWDTLNHLLFISSCLYKLNLKSVAQITLSILAMIISTSLIIAAIIFIASANHKVTPTTAIIQDATSQIKNTPPSKPNNDFHFEVFNFVPCSICSNNPTCWAICKRIPNKKPGKKTTTKPTKKP

SEQ ID NO: 4 - protein, 297 aa, synthetic construct
MSKNKDQRTAKTLERTWDTLNHLLFISSCLYKLNLKSVAQITLSILAMIISTSLIIVAIIFIASANHKITSTTTIIQDATNQIKNTTPTYLTQNPQLGISPSNPSDITSLITTILDSTTPGVKSTLQSTTVGTKNTTTTQAQPNKPTTKQRQNKPPSKPNNDFHFEVFNFVPCSICSNNPTCWAICKRIPNKKPGKRTTTKPTKKPTPKTTKKGPKPQTTKSKEAPTTKPTEEPTINTTKTNIITTLLTSNTTRNPELTSQMETFHSTSSEGNPSPSQVSITSEYPSQPSSPPNTPR

SEQ ID NO: 5 - protein, 89 aa, synthetic construct
MSKNKDQRTAKTLERTWDTLNHLLFISSCLYKLNLKSVAQITLSILAMIISTSLIIVAIIFIASANHKITSTTTIIQDATNQIKNTTPT

SEQ ID NO: 6 - protein, 89 aa, synthetic construct
MSKNKDQRTAKTLERTWDTLNHLLFISSCLYKLNLKSVAQITLSILAMIISTSLIIAAIIFIASANHKVTPTTAIIQDATSQIKNTPPS

SEQ ID NO: 7 - protein, 22 aa, synthetic construct
KVTPTTAIIQDATSQIKNTPPS

SEQ ID NO: 8 - protein, 44 aa, synthetic construct
TKQRQNKPPSKPNNDFHFEVFNFVPCSICSNNPTCWAICKRIPN

SEQ ID NO: 9 - protein, 44 aa, synthetic construct
TSQIKNTPPSKPNNDFHFEVFNFVPCSICSNNPTCWAICKRIPN

SEQ ID NO: 10 - protein, 10 aa, synthetic construct
TSQIKNTPPS

SEQ ID NO: 11 - DNA, 302 bp, synthetic construct
  source 1..302: mol_type = other DNA
ggggcaaatgcaaacatgataatatcaacatctttgattatcgcggcaatcatattcatcgcctccgccaatcacaaagtgacacctacgacggcgattattcaagatgcgacttcacaaataaagaatacaccgccaagcaagcccaataacgattttcactttgaagtatttaacttcgtaccctgctcaatttgtagtaataaccccacttgctgggctatttgtaaacgcatcccaaataaaaagcctggtaagaagacaactacgaagcccacaaaaaaaccttagttacttaaaaa

SEQ ID NO: 12 - DNA, 443 bp, synthetic construct
  source 1..443: mol_type = other DNA
ggggcaaatgcaaacatgtcaaagaataaggatcaaagaactgcgaagaccctcgaacggacttgggatacgctgaatcatttgctctttatatcttcctgcctctataagctgaacctcaagtctgtggcacaaattacgctgagtattctggccatgataatatcaacatctttgattatcgcggcaatcatattcatcgcctccgccaatcacaaagtgacacctacgacggcgattattcaagatgcgacttcacaaataaagaatacaccgccaagcaagcccaataacgattttcactttgaagtatttaacttcgtaccctgctcaatttgtagtaataaccccacttgctgggctatttgtaaacgcatcccaaataaaaagcctggtaagaagacaactacgaagcccacaaaaaaaccttagttacttaaaaa

SEQ ID NO: 13 - DNA, 15296 bp, synthetic construct
  source 1..15296: mol_type = other DNA
acgcgaaaaaatgcgtacaacaaacttgcataaaccaaaaaaatggggcaaataagaatttgataagtaccacttaaatttaactcccttgcttagcgatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgagtattcaattatagttattaaaaacttaacagaagacaaaaatggggcaaataagaatttgataagtaccacttaaatttaactcccttgcttagcgatgggcagcaattcattgagtatgataaaagttagattacaaaatttgtttgacaatgatgaagtagcattgttaaaaataacatgctatactgataaattaatacatttaactaacgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagattacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggacgtccatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaattagggcccaacaatgaactaggatatcaagactaacgacgtccatggggcaaatgcaaacatgataatatcaacatctttgattatcgcggcaatcatattcatcgcctccgccaatcacaaagtgacacctacgacggcgattattcaagatgcgacttcacaaataaagaatacaccgccaagcaagcccaataacgattttcactttgaagtatttaacttcgtaccctgctcaatttgtagtaataaccccacttgctgggctatttgtaaacgcatcccaaataaaaagcctggtaagaagacaactacgaagcccacaaaaaaaccttagttacttaaaaacatattatcacaaaaggccttgaccaaccgcggagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaagaaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcagagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaaaatgtaaagttcaatcgaatcgagtattttgtgacacaatgtacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcatgataactactataattatagtgattatagtaatattgttatcattaattgctgttggactgctcctatactgtaaggccagaagcacaccaatcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaatgccagtcgaccttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacgctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatgattggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccactcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactgtggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

(19) United States
(12) Patent Application Publication
(10) Pub. No.: US 2026/0115274 A1
(43) Pub. Date: Apr. 30, 2026

(54) RSV Vaccines with Truncated G-Protein Mucin Domains

(71) Applicants: Emory University, Atlanta, GA (US); Children's Healthcare of Atlanta, Inc., Atlanta, GA (US); Butler University, Indianapolis, IN (US)

(72) Inventors: Christina A. Rostad, Atlanta, GA (US); Evan J. Anderson, Atlanta, GA (US); Stacey Lapp, Atlanta, GA (US); Larry J. Anderson, Atlanta, GA (US); Christopher C. Stobart, Indianapolis, IN (US); Molly K. Roe, Indianapolis, IN (US)

(21) Appl. No.: 19/116,944

(22) PCT Filed: Sep. 29, 2023
(86) PCT No.: PCT/US2023/075562

§ 371 Date: Mar. 28, 2025

Related U.S. Application Data
(60) Provisional application No. 63/411,251, filed on Sep. 29, 2022.

(51) Int. Cl. A61K 39/155; A61K 39/00; C07K 14/135; C12N 7/00; C12N 15/11; C12N 15/63

(52) U.S. Cl. CPC A61K 39/155; C07K 14/135; C12N 7/00; C12N 15/11; C12N 15/63; A61K 2039/5254; A61K 2039/53; A61K 2039/575; C12N 2760/18521; C12N 2760/18522; C12N 2760/18534; C12N 2760/18561

(57) ABSTRACT

The disclosure relates to Respiratory Syncytial Virus (RSV) vaccine compositions having truncated mucin domains in the G-protein. In certain embodiments, this disclosure relates to virus particles, virus-like particles, virosomes, nucleic acids, vectors, or attenuated live RSV vaccines for uses reported herein. In certain embodiments, this disclosure relates to methods of vaccinating, treating, or preventing RSV infections by administering to a subject in need thereof an effective amount of a composition disclosed herein.

### CROSS-REFERENCE TO RELATED APPLICATIONS

This application claims the benefit of U.S. Provisional Application No. 63/411,251 filed Sep. 29, 2022. The entirety of this application is hereby incorporated by reference for all purposes.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 21191PCT.xml. The XML file is 29 KB, was created on Sep. 28, 2023, and is being submitted electronically via the USPTO patent electronic filing system.

### BACKGROUND

Human respiratory syncytial virus (RSV) causes acute lower respiratory infections. It is a major cause of hospital visits for premature babies and newborns. RSV infections also pose a threat for the elderly and immune compromised. Palivizumab is a humanized chimeric antibody that binds the RSV fusion protein (RSV F) that is clinically approved for prevention of serious lower respiratory tract disease caused by RSV in certain high-risk infants. Palivizumab has limited efficacy and sometimes causes allergic reactions. Thus, there is a need to identify additional RSV therapies.

Vaccines are typically killed (inactivated) or weakened (attenuated) versions of a live viral strain. Kim et al. report that administration of a formalin-inactivated RSV vaccine was not sufficiently effective and primed for enhanced disease. Am J Epidemiol 89, 422-434 (1969). Attenuated RSV vaccine candidates face significant safety hurdles, and the development of pediatric RSV live-attenuated vaccine (LAV) strains that are sufficiently attenuated and immunogenic have been elusive. See Collins et al. Progress in understanding and controlling respiratory syncytial virus: still crazy after all these years. Virus Res, 2011, 162, 80-99.

Zhang et al. report vaccination to induce antibodies blocking the CX3C-CX3CR1 interaction of respiratory syncytial virus G protein reduces pulmonary inflammation in mice. J Virol, 2010, 1148-1157.

Chirkova et al. report respiratory syncytial virus G protein CX3C motif impairs human airway epithelial and immune cell responses. J Virol, 2013, 87:13466-13479.

Boyoglu-Barnum et al. report prophylaxis with a respiratory syncytial virus (RSV) anti-G protein monoclonal antibody shifts the adaptive immune response to RSV rA2-line19F infection from Th2 to Th1. J Virol, 2014, 88:10569-10583.

Boyoglu-Barnum et al. report an anti-G protein monoclonal antibody treats RSV disease more effectively than an anti-F monoclonal antibody. Virology, 2015, 483:117-125.

Bakre et al. report the central conserved region of RSV G protein modulates host miRNA expression and alters the cellular response to infection. Vaccines, 2017, 5 (16): 15 pages.

Caidi et al. report anti-RSV G monoclonal antibodies reduce lung inflammation and viral lung titers when delivered therapeutically. Antiviral Research, 2018, 154:149-157.

Ha et al. report mutation of respiratory syncytial virus G protein's CX3C motif attenuates infection in human airway epithelial cells. Vaccines, 2019, 7 (69): 16 pages.

Brakel et al. report codon-optimization of the RSV G protein expressed in a vesicular stomatitis virus vector improves immune responses. Virology, 2022, 575:101-110.

See also WO 2016/040556 and U.S. Pat. Nos. 11,471,524, 11,235,050, 10,792,356, 10,626,378, and 10,232,032.

References cited herein are not an admission of prior art.

### SUMMARY

The disclosure relates to Respiratory Syncytial Virus (RSV) vaccine compositions having truncated mucin domains in the G-protein. In certain embodiments, this disclosure relates to recombinant attenuated RSV viruses having truncated C-terminal and/or N-terminal mucin domains, e.g., RSV A2-line19F-G155 and A2-line19F-G155S. In certain embodiments, this disclosure relates to virus particles, virus-like particles, virosomes, nucleic acids, vectors, or attenuated live RSV vaccines for uses reported herein. In certain embodiments, this disclosure relates to methods of vaccinating, treating, or preventing RSV infections by administering to a subject in need thereof an effective amount of a composition disclosed herein.

In certain embodiments, this disclosure relates to recombinant RSV encoding G proteins wherein mucin domains or segments thereof are deleted. In certain embodiments, 10, 20, 30, 40, 50, 60, 70, 80, 90, or 100 or more continuous amino acids in the N-terminal mucin like domain 1 are deleted, e.g., spanning amino acid positions 70-154 or 135-154 of the G protein (e.g. as in SEQ ID NO: 4) and/or 10, 20, 30, 40, 50, 60, 70, 80, 90, or 100 or more amino acids in the C-terminal mucin like domain II are deleted, e.g., spanning amino acid position 207-297 of the G protein (e.g. as in SEQ ID NO: 4). In certain embodiments, the C-terminal amino acids 1-47 of the G protein are deleted. Embodiments of this disclosure include any of the nucleic acid or protein sequences disclosed herein or variants thereof.

In certain embodiments, this disclosure relates to recombinant RSV G proteins comprising the amino acid sequence of AIIFIASANHKVTPTTAIIQDATSQIKNTPPS (SEQ ID NO: 1), KVTPTTAIIQDATSQIKNTPPS (SEQ ID NO: 7), and/or TSQIKNTPPS (SEQ ID NO: 10) variants thereof.

In certain embodiments, this disclosure relates to recombinant RSV G proteins comprising the amino acid sequence of

MIISTSLIIAAIIFIASANHKVTPTTAIIQDATSQIKNTPPSKPNNDFHFEVFNFVPCSI CSNNPTCWAICKRIPNKKPGKKTTTKPTKKP (SEQ ID NO: 2) (G155S) or variants thereof.

In certain embodiments, this disclosure relates to recombinant RSV G proteins comprising the amino acid sequence of

MSKNKDQRTAKTLERTWDTLNHLLFISSCLYKLNLKSVAQITLSILAMIISTSLIIA AIIFIASANHKVTPTTAIIQDATSQIKNTPPSKPNNDFHFEVFNFVPCSICSNNPTCWAICKR IPNKKPGKKTTTKPTKKP (SEQ ID NO: 3) (G155) or variants thereof.

In certain embodiments, this disclosure relates to recombinant RSV G proteins comprising the amino acid sequence of KVTPTTAIIQDATSQIKNTPPS (SEQ ID NO: 7) or variants thereof.

In certain embodiments, this disclosure relates to recombinant RSV G proteins comprising the amino acid sequence of TSQIKNTPPS (SEQ ID NO: 10) or variants thereof.

In certain embodiments, this disclosure relates to nucleic acids and vectors encoding recombinant RSV G proteins as reported herein. In certain embodiments, the nucleic acid is RNA or DNA in operable combination with a promoter for expression of the RSV G protein.

In certain embodiments, this disclosure relates to a live attenuated RSV vaccines comprising or encoding an RSV G protein as provided herein.

In certain embodiments, this disclosure relates to host cells and other expression systems comprising a nucleic acid or vector encoding an RSV G protein as reported herein.

In certain embodiments, this disclosure contemplates immunogenic compositions comprising nucleic acids or vectors encoding an RSV G protein as provided herein and a pharmaceutically acceptable excipient.

In certain embodiments, this disclosure contemplates immunogenic compositions of virus particles, virus like particles, virosomes, or cells comprising or having nucleic acids encoding an RSV G protein as provided herein and a pharmaceutically acceptable excipient.

In certain embodiments, this disclosure relates to methods for vaccinating a subject for RSV or inducing an effective immune response against RSV infection in a subject, comprising administering to the subject an immunologically effective dose of the immunogenic composition against RSV as reported herein. In certain embodiments, the subject is a human patient, a pregnant mother, infant, child, adult, or newborn less than one year old.

### BRIEF DESCRIPTION OF THE SEVERAL VIEWS OF THE DRAWINGS

FIG. 1A illustrates a nucleic acid sequence (SEQ ID NO: 11) that encodes a G protein (G155S) (SEQ ID NO: 2)

MIISTSLIIAAIIFIASANHKVTPTTAIIQDATSQIKNTPPSKPN
NDFHFEVFNFVPCSICSNNPTCWAICKRIPNKKPGKKTTTKPTKK
P.

FIG. 1B illustrates a nucleic acid sequence (SEQ ID NO: 12) that encodes a G protein (G155) (SEQ ID NO: 3),

MSKNKDQRTAKTLERTWDTLNHLLFISSCLYKLNLKSVAQITLSI
LAMIISTSLIIAAIIFIASANHKVTPTTAIIQDATSQIKNTPPSK
PNNDFHFEVFNFVPCSICSNNPTCWAICKRIPNKKPGKKTTTKPT
KKP.

FIG. 1C illustrates a sequence alignment using BLASTP (NCBI) of an RSV G reference sequence (SEQ ID NO: 4) Query (Q) having UniProtKB/Swiss-Protein Accession number P27022.1 and truncated RSV G having SEQ ID NO: 3. A difference is that truncated RSV G has a variant sequence AAIIFIASANHKVTPTTAIIQDATSQIKNTPPS (SEQ ID NO. 1). Amino acids 1-89 of Q (SEQ ID NO 5) are compared to those of the Subject(S) sequence (SEQ ID NO: 6). Notable changes were in SEQ ID NO: 7. Amino acids 148-191 of Q (SEQ ID NO: 8) are compared to those of S (SEQ ID NO: 9). Notable changes were in SEQ ID NO: 10.

FIG. 2A shows a schematic of RSV wild-type A2-line19. A2-line19F is wild-type A2 virus with a substituted thermostable fusion protein gene from the line 19 strain. Numbers represent amino acid residues in wild-type A2 G protein. Abbreviations: aa, amino acid; CCD, central conserved domain; COOH, C-terminal domain; CT, cytoplasmic tail; HBD, heparin binding domain; Met, methionine; NH2, N-terminal domain; RSV, respiratory syncytial virus; TM, transmembrane domain; wtG, wild-type G.

FIG. 2B shows a schematic of G-mutant vaccines. A2-line19F-G155 has a deletion that removes the mucin domains from G protein. A2-line19F-G155S additionally has removal of a segment of the G protein transmembrane domain sequence. Thus, this strain expresses a G protein limited to the Central Conserved Domain (CCD), which is exclusively secreted (no substantial membrane-bound G because it is lacking the cytoplasmic tail (CT) and portion of the transmembrane (TM) domain.

FIG. 3 shows data on the immunogenicity and efficacy of G-mutant vaccines in BALB/c mice. Mice were primed and boosted on days 1 and 29, respectively. Binding and neutralizing antibody responses were measured on days 0, 28, and 59 by (top left) RSV prefusion F ELISA, and (top right) neutralizing antibodies to A2-line19F in HEp-2 cells. Sera from groups of mice were pooled and analyzed in 2 replicates in duplicate for ELISAs, which are expressed as log 2 (end-point titers) for ELISAs and geometric means of the log (EC₅₀) for neutralization assays. On day 60, mice were challenged intranasally with 10⁶ FFU A2-line 19F. Four days later, lungs were harvested, and virus titrated by either (bottom left) live-virus FFU assay or (bottom right) RT-PCR. Data represent the geometric mean titer (±geometric SD) viral lung titer in log 10 (FFU/g lung). Data represent the mean (±SD) delta-delta cycle threshold (AACt) value between GAPDH and RSV M gene, normalized to mock. EC₅₀, 50% maximum effective concentration; ELISA, enzyme-linked immunosorbent assay; FFU, fluorescent focus unit; GAPDH, glyceraldehyde-3-phosphate dehydrogenase; RSV, respiratory syncytial virus; RT-PCR, real-time polymerase chain reaction. The dashed lines represent the lower limits of detection of the assays.

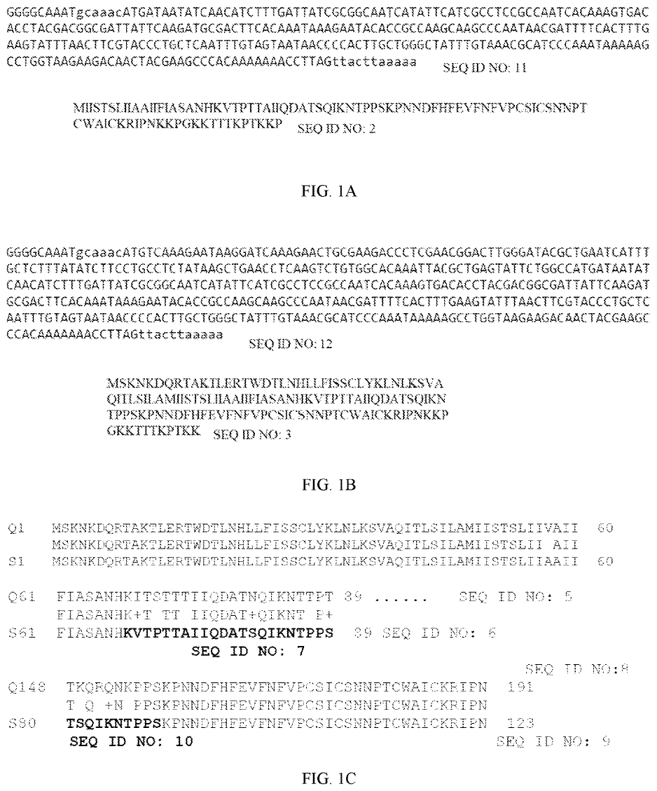

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

A “nucleic acid” refers to and DNA- or RNA-molecule and is used synonymously with polynucleotide. Wherever herein reference is made to a nucleic acid or nucleic acid sequence encoding a particular protein and/or peptide, said nucleic acid preferably also comprises regulatory sequences allowing in a suitable host, e.g., a human being, its expression, i.e., transcription and/or translation of the nucleic acid sequence encoding the particular protein or peptide.

“Amino acid sequence” is defined as a sequence composed of any one of the 20 naturally appearing amino acids, amino acids which have been chemically modified, or composed of synthetic amino acids. The terms “protein” and “peptide” refer to compounds comprising amino acids joined via peptide bonds and are used interchangeably. An “amino acid sequence” can be deduced from the nucleic acid sequence encoding the protein. Furthermore, unless the context demands otherwise, the term “peptide” and “polypeptide” and “protein” are used interchangeably to refer to amino acids in which the amino acid residues are linked by covalent peptide bonds or alternatively (e.g., where post-translational processing has removed an internal segment) by covalent disulfide bonds, etc. The ammo acid chains can be of any length and comprise at least two amino acids or at least three amino acids, they can include domains of proteins or full-length proteins. Unless otherwise stated the terms peptide, polypeptide, and protein also encompass various modified forms thereof, including but not limited to glycosylated forms, phosphorylated forms, etc.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a range of amino acids expressly specified in the claim. In certain embodiments, the disclosure contemplates that the “N-terminus of a peptide consists of an amino acid sequence,” which refers to the N-terminus of the peptide having the exact number of amino acids in the sequence and not more or having not more than a range of amino acids specified in the claim; however, the C-terminus may be connected to additional amino acids, e.g., as part of a larger peptide. Similarly, the disclosure contemplates that the “C-terminus of a peptide consists of an amino acid sequence,” which refers to the C-terminus of the peptide having the exact number of amino acids in the sequence and not more or having not more than a range of amino acids specified in the claim; however, the N-terminus may be connected to additional amino acids, e.g., as part of a larger peptide.

The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

As used herein, a “heterologous” gene, nucleic acid, promoter, antigen, protein, etc., is understood to be referring to a nucleic acid or amino acid sequence which is not present in naturally occurring nucleic acids or proteins.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular, or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo (e.g., host cell) and in vitro (cell free) systems. Systems for recombinant protein expression typically utilize somatic cells transfecting with a DNA or mRNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids, and nucleotides. In the presence of an expression vector, these extracts and components can synthesize proteins of interest. Cell-free systems typically do not contain proteases and enable labeling of the protein with modified amino acids.

A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence of an antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-B-D-galactoside). If recombinant insertion inactivates the lac-z-gene, then the resulting colonies are colorless. Additional contemplated selectable markers include any genes that impart antibacterial resistance or express a fluorescent protein. Examples include, but are not limited to, the following genes: ampr, camr, tetr, blasticidinr, neor, hygr, abxr, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose: galactose-1-phosphate uridyltransferasel (galT), feedback-insensitive a subunit of anthranilate synthase (OASAID), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, the disclosure relates to recombinant polypeptides comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a polypeptide disclosed herein or chimeric protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

“Variant” refers to a polypeptide or polynucleotide that differs from a reference polypeptide or polynucleotide and retains essential properties. A typical variant of a polypeptide differs in amino acid sequence from another, reference polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions). A substituted or inserted ammo acid residue may or may not be one encoded by the genetic code. A variant of a polypeptide may be naturally occurring such as an allelic variant, or it may be a variant that is not known to occur naturally.

Modifications and changes can be made in the structure of the peptides of this disclosure and still result in a molecule having similar characteristics as the peptide (e.g., a conservative amino acid substitution). For example, certain amino acids can be substituted for other amino acids in a sequence without appreciable loss of activity. Because it is the interactive capacity and nature of a peptide that defines the biological functional activity of the peptide, certain ammo acid sequence substitutions can be made in a peptide sequence and nevertheless obtain a peptide with like properties. Amino acid substitutions are generally based on the relative similarity of the ammo acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala to Gly or Ser), (Arg to Lys), (Asn to Gln or His), (Asp to Glu, Cys, or Ser), (Gln to Asn), (Glu to Asp), (Gly to Ala), (His to Asn, Gln), (Leu to Ile or Val), (Lys to Arg), (Met to Leu, Tyr), (Ser to Thr), (Thr to Ser), (Trp to Tyr), (Tyr to Trp or Phe), and (Val to Ile or Leu). Embodiments of this disclosure thus contemplate functional or biological equivalents of a peptide as set forth above. In certain embodiments, peptide variants are those having greater than 50%, 60%, 70%, 80%, 90%, 95%, 96%, 97%, 97%, and 99% sequence identity to the peptide of interest.

“Identity,” as known in the art, is a relationship between two or more peptide sequences, as determined by comparing the sequences. In the art, “identity” also refers to the degree of sequence relatedness between peptides as determined by the match between strings of such sequences. “Identity” and “similarity” can be readily calculated by known methods. Preferred methods to determine identity are designed to give the largest match between the sequences tested. In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. Methods to determine identity and similarity are codified in publicly available computer programs. The percent identity between two sequences can be determined by using analysis software (Sequence Analysis Software Package of the Genetics Computer Group, Madison, Wis) that incorporates the Needelman & Wunsch (J Mol Biol, 48 443-453, 1970) algorithm (e.g., NBLAST and XBLAST). The US National Institutes of Health has a website that provides for aligning two protein sequences using BLASTP, which was used to create certain sequence alignments disclosed herein. The default parameters may be used to determine the identity for the peptides of the present disclosure.

In general, homologous peptides of the present disclosure are characterized as having one or more amino acid substitutions, deletions, and/or additions.

In certain embodiments, peptide variants are those having 1 amino acid substitution. In certain embodiments, peptide variants are those having 2 amino acid substitutions. In certain embodiments, peptide variants are those having 3 amino acid substitutions. In certain embodiments, peptide variants are those having 4 amino acid substitutions. In certain embodiments, peptide variants are those having 5 amino acid substitutions. In certain embodiments, peptide variants are those having 6 amino acid substitutions. In certain embodiments, peptide variants are those having 7 amino acid substitutions. In certain embodiments, peptide variants are those having 8 amino acid substitutions. In certain embodiments, peptide variants are those having 9 amino acid substitutions. In certain embodiments, peptide variants are those having 10 amino acid substitutions. In certain embodiments, peptide variants are those having 1 amino acid substitution, deletion, and/or addition. In certain embodiments, peptide variants are those having 2 amino acid substitutions, deletions, and/or additions. In certain embodiments, peptide variants are those having 3 amino acid substitutions, deletions, and/or additions. In certain embodiments, peptide variants are those having 4 amino acid substitutions, deletions, and/or additions. In certain embodiments, peptide variants are those having 5 amino acid substitutions, deletions, and/or additions. In certain embodiments, peptide variants are those having 6 amino acid substitutions, deletions, and/or additions. In certain embodiments, peptide variants are those having 7 amino acid substitutions, deletions, and/or additions. In certain embodiments, peptide variants are those having 8 amino acid substitutions, deletions, and/or additions. In certain embodiments, peptide variants are those having 9 amino acid substitutions, deletions, and/or additions. In certain embodiments, peptide variants are those having 10 amino acid substitutions, deletions, and/or additions.

In certain embodiments, peptide variants are those having 1 conserved amino acid substitution. In certain embodiments, peptide variants are those having 2 conserved amino acid substitutions. In certain embodiments, peptide variants are those having 3 conserved amino acid substitutions. In certain embodiments, peptide variants are those having 4 conserved amino acid substitutions. In certain embodiments, peptide variants are those having 5 conserved amino acid substitutions. In certain embodiments, peptide variants are those having 6 conserved amino acid substitutions. In certain embodiments, peptide variants are those having 7 conserved amino acid substitutions. In certain embodiments, peptide variants are those having 8 conserved amino acid substitutions. In certain embodiments, peptide variants are those having 9 conserved amino acid substitutions. In certain embodiments, peptide variants are those having 10 conserved amino acid substitutions.

By “conservative amino acid substitution,” as used herein, is meant replacement, in an amino acid sequence, of one amino acid with another amino acid of the same family of amino acids, as based on the chemical nature of their side chains. Genetically encoded amino acids can be divided into four families: acidic (aspartate, glutamate); basic (lysine, arginine, histidine); nonpolar (alanine, valine, leucine, isoleucine, praline, phenylalanine, methionine, tryptophan); and uncharged polar (glycine, asparagine, glutamine, cysteine, serine, threonine, tyrosine). Phenylalanine, tryptophan, and tyrosine are sometimes grouped as aromatic amino acids. In similar fashion, the amino acids can also be separated into the following groups: acidic (aspartate, glutamate); basic (lysine, arginine, histidine); aliphatic (glycine, alanine, valine, leucine, isoleucine, serine, threonine), with serine and threonine optionally grouped separately as aliphatic-hydroxyl; aromatic (phenylalanine, tyrosine, tryptophan); amide (asparagine, glutamine); and sulfur-containing (cysteine, methionine).

The term “virus particles” as used herein refers to virus-like particles, virosomes, or particles created and/or isolated by a replicating virus, e.g., chimeric virus, attenuated virus.

The term “virus-like particle” (VLPs) as used herein refers to a membrane-surrounded viral core structure having viral envelope proteins. Further, viral core proteins are located within the membrane of the VLP. Typically, a virus-like particle does not carry genetic information encoding for the proteins of the virus-like particle. In general, virus-like particles lack the viral genome and, therefore, are noninfectious. Also, virus-like particles can often be produced in large quantities by heterologous expression and can be purified. Some virus-like particles may contain a nucleic acid distinct from their genome. Typically, a virus-like particle is non replicative and noninfectious lacking all or part of the viral genome, in particular the replicative and infectious components of the viral genome. The term “virosome” as used herein refers to a virus particle that is similar to a virus-like particle, except that a virosome does not contain a viral core protein.

The terms “chimeric respiratory syncytial virus” or “chimeric RSV” refer to a nucleic acid that contains sufficient RSV genes to allow the genome or antigenome to replicate in host cells and the nucleic acid sequence is altered to include at least one nucleic acid segment that is not structurally the same a natural RSV strain, i.e., such that the nucleotide sequence of the RSV strain does not occur naturally over the whole RSV genome. A chimeric respiratory syncytial virus includes an RSV gene wherein the codons are altered to be different from those naturally occurring gene even though the RSV produces a polypeptide with an identical amino acid sequence to those naturally expressed. Different strains of RSV will have different nucleotide sequences and express proteins with different amino acid sequences that have similar functions. Thus, a chimeric RSV includes an RSV gene wherein one or more genes from one strain are replaced from genes in alternative or second strain such that the nucleic acid sequence of the entire RSV genome is not identical to an RSV found in nature. In certain embodiments, the chimeric RSV includes those strains where nucleic acids are deleted after a codon for starting translation in order to truncate the proteins expression, provided such truncation pattern for the genome is not found in naturally occurring RSV. In certain embodiments, the chimeric RSV includes those which are infectious but cannot replicate in a human subject. In certain embodiments, the chimeric RSV includes those which are infectious and can replicate in a human subject.

A “subject” refers to any animal, preferably a human patient, livestock, or domestic pet.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, an “RSV G” protein, or like terms refers to an attachment RSV glycoprotein G and all known variants or substantial fragments thereof. One example has the amino acid sequence of

MSKNKDQRTAKTLERTWDTLNHLLFISSCLYKLNLKSVAQITLSILAMIISTSLIIV AIIFIASANHKITSTTTIIQDATNQIKNTTPTYLTQNPQLGISPSNPSDITSLITTILDSTTPGV KSTLQSTTVGTKNTTTTQAQPNKPTTKQRQNKPPSKPNNDFHFEVFNFVPCSICSNNPTC WAICKRIPNKKPGKRTTTKPTKKPTPKTTKKGPKPQTTKSKEAPTTKPTEEPTINTTKTNII TTLLTSNTTRNPELTSQMETFHSTSSEGNPSPSQVSITSEYPSQPSSPPNTPR (SEQ ID NO: 4, UniProtKB/Swiss-Protein Accession number P27022.1) which is an RSV G protein sequence wherein the N-terminal Methionine (M) is position one. Other examples include those designated by Accession numbers P03423.1, CAA51765.1, AAD02944.1, CAA83874.1, AAD02941.1, CAA83870.1, P27021.1, AEO45938.1, CAA51761.1, CAA83871.1, AAU43727.1, AEO45918.1, AEO45888.1, AEO45878.1, AEO45849.1, AEC32086.1, AEQ98767.1, AEQ63333.1, CAA83875.1, AEO45908.1, AEO45948.1, AEO45898.1, AEO45928.1, AAC36327.1, ACO83296.1, AEO45829.1, AEQ66845.1, ACI03570.1, AEO45868.1, P20895.2, AAD02943.1, AAX23993.1, AEQ66853.1, AEO45839.1, AEJ87998.1, and AEJ88006.1.

Throughout the present patent specification, when reference is made to specific amino acid residues or specific amino acid regions, e.g., in the RSV G, by referring to their amino residue number or numbers, and unless otherwise stated, the numbering is based on the RSV amino acid sequences provided herein in the sequence listing and in the Figures. It should be noted, and one of skill in the art will understand, that different RSV sequences may have different numbering systems, for example, if there are additional amino acid residues added or removed as compared to SEQ ID NO: 4. As such, it is to be understood that when specific amino acid residues are referred to by their number, the description is not limited only to amino acids located at precisely that numbered position when counting from the beginning of a given amino acid sequence, but rather that the equivalent/corresponding amino acid residue in any and all RSV G sequences is intended—even if that residue is not at the same precise numbered position, for example if the RSV sequence is shorter or longer than SEQ ID NO: 4, or has insertions or deletions as compared to SEQ ID NO: 4. One of skill in the art can readily determine what is the corresponding/equivalent amino acid position to any of the specific numbered residues recited herein, for example by aligning a given RSV G sequence to SEQ ID NO: 4.

In certain embodiments, this disclosure relates to recombinant RSV encoding G proteins wherein mucin domains or segments thereof are deleted. In certain embodiments, 10, 20, 30, 40, 50, 60, 70, 80, 90, or 100 or more continuous amino acids in the mucin like domain 1 are deleted, e.g., the amino acid spanning position at about 70-154 or 135-154 of the G protein (e.g. as in SEQ ID NO: 4) and/or 10, 20, 30, 40, 50, 60, 70, 80, 90, or 100 or more amino acids in the C-terminal mucin like domain II are deleted, e.g., the amino acid spanning at about 207-297 of the G protein (e.g. as in SEQ ID NO: 4). In certain embodiments, the C-terminal amino acids 1-47 of the G protein are deleted.

In certain embodiments, this disclosure relates to recombinant RSV G proteins comprising the amino acid sequence of AIIFIASANHKVTPTTAIIQDATSQIKNTPPS (SEQ ID NO: 1).

In certain embodiments, this disclosure relates to recombinant RSV G proteins comprising the amino acid sequence of

(SEQ ID NO: 2)
MIISTSLIIAAIIFIASANHKVTPTTAIIQDATSQIKNTPPSKPN
NDFHFEVFNFVPCSICSNNPTCWAICKRIPNKKPGKKTTTKPTKK
P
(G155S).

In certain embodiments, this disclosure relates to recombinant RSV G proteins comprising the amino acid sequence of

(SEQ ID NO: 3)
MSKNKDQRTAKTLERTWDTLNHLLFISSCLYKLNLKSVAQITLSI
LAMIISTSLIIAAIIFIASANHKVTPTTAIIQDATSQIKNTPPSK
PNNDFHFEVFNFVPCSICSNNPTCWAICKRIPNKKPGKKTTTKPT
KKP
(G155).

In certain embodiments, this disclosure relates to a protein having the amino acid sequence of SEQ ID NO: 1, 2, 3, 7, and/or 10 or variant proteins with greater than 80%, 85%, 90%, 95%, 96%, 97%, 98%, or 99% sequence identity. In certain embodiments, the variants are conserved substitutions.

In certain embodiments, this disclosure relates to recombinant RSV G proteins comprising one or more G mutations V57A, 169V, S71P, T74A, N81S, T87P, T89S, K149S, R151I, Q152K, K154T, or combinations thereof.

The RSV fusion protein (F) is a surface glycoprotein used to fuse to target cell membranes. The RSV fusion protein exists in a pre-fusion conformation. The F protein is an RSV immunogen. Anti-RSV neutralizing antibodies are often directed against the F protein in the pre-fusion conformation. Mutations for stabilizing the prefusion F protein include S155C and S290C for disulfide formation (DS), S190F and V207L for cavity filling (Cav1), and D486H, E487Q, F488W, and D489H to form stable RSV F trimers (TriC). See McLellan et al. Structure-Based Design of a Fusion Glycoprotein Vaccine for Respiratory Syncytial Virus, Science. 2013, 342 (6158): 592-598.

In certain embodiments, this disclosure contemplates the use of RSV G proteins disclosed herein in combination with RSV F proteins with prefusion stabilizing construction/mutations.

In certain embodiments, this disclosure relates to nucleic acids encoding the recombinant RSV protein, such as G protein and/or F protein as reported herein in operable combination with a heterologous promoter.

In certain embodiments, this disclosure relates to vectors comprising a nucleic acid as reported herein or encoding the recombinant RSV G protein as reported herein.

In certain embodiments, this disclosure relates to attenuated RSV strains comprising a nucleic acid encoding an RSV G protein and/or RSV F protein as reported herein.

In certain embodiments, the nucleic acid is mRNA, RNA, or DNA.

In certain embodiments, this disclosure relates to a recombinant plasmid or bacterial artificial chromosomes comprising a nucleic acid encoding an RSV G protein disclosed herein.

In certain embodiments, the disclosure relates to live attenuated viruses, chimeric RSVs, or nucleic acids encoding G proteins as reported herein that are contain in a plasmid or bacterial artificial chromosomes.

In certain embodiments, the RSV comprises the nucleic acid sequence of SEQ ID NO: 13.

In certain embodiments, this disclosure relates to virus particles/virus like particles/virosomes comprising any of the recombinant RSV G proteins as reported herein. In certain embodiments, the virus particles/virus like particles/virosomes can further include an F protein, a small hydrophobic (SH) protein, and/or a matrix (M) protein of RSV. In certain embodiments, the virus particles/virus like particles/virosomes also include lipids and proteins extracted from the membrane of an RSV strain (e.g., a chimeric RSV strain including an RSV strain in which the F protein is replaced with an RSV Line 19 F protein (e.g., RSV strain A2)). In certain embodiments, the F protein be substantially in the pre-fusion conformation.

In certain embodiments, this disclosure relates to methods of vaccinating against or treating for an RSV infection comprising administering an effective amount of a vaccine or pharmaceutical composition comprising RSV G protein, viral particle/virus like particle/virosome, nucleic acid, attenuated RSV strain, or vector as reported herein to a subject in need thereof. In certain embodiments, the subject does not have, but is at risk of developing, RSV infection.

In certain embodiments, compositions are for use as medicaments, for inducing an immune response to RSV, for use in methods of preventing or treating RSV infection, and/or for vaccinating a subject against RSV.

In certain embodiments, this disclosure contemplates methods for producing an immunogenic composition against RSV comprising providing a host cell culture; inoculating the host cell culture with a nucleic acid encoding an RSV G protein as provided herein; incubating the cell culture with the RSV; harvesting RSV following the incubation step; and formulating the harvested RSV into an immunogenic composition against RSV.

In certain embodiments, particles or virosomes can further include one or more lipids, phospholipids, a phosphatidylcholine (PC), a phosphatidylethanolamine (PE) species, a sterol or sterol derivative (e.g., cholesterol), 1,2-dioleoyl-sn-glycero-3-phosphocholine (DOPC), 1,2-dioleoyl-sn-glycero-3-phosphoetanolamine (DOPE), 1,2-dipalmitoleoyl-sn-glycero-3-phosphoethanolamine (PPPE), and 1-palmitoyl-2-linoleoyl-sn-glycero-3-phosphoethanolamine (PLPE), or combinations thereof. In certain embodiments, the virus particles/virus like particles/virosomes can also include an adjuvant such as, e.g., a saponin, PHAD (phosphorylated hexa-acyl disaccharide), 3-D-PHAD (3-O-desacyl derivative of phosphorylated hexa-acyl disaccharide), 3-O-D MPLA (3-O-desacyl derivative of monophosphoryl lipid A), or MPLA (monophosphoryl lipid A).

In certain embodiments, this disclosure relates to vaccines or pharmaceutical compositions comprising an RSV G protein, viral particle/virus like particle, nucleic acid, attenuated RSV virus, or vector as reported herein.

### Vaccination Methods

In certain embodiments, this disclosure relates to methods of vaccinating against or treating for an RSV infection comprising administering an effective amount of a vaccine or pharmaceutical composition comprising RSV G protein, viral particle/virus like particle/virosome, nucleic acid, attenuated RSV strain, or vector as reported herein to a subject in need thereof.

In certain embodiments, the vaccine or pharmaceutical composition is administered intranasally, intradermally, or intramuscularly.

In certain embodiments, this disclosure relates to methods of vaccinating against or treating for an RSV infection comprising administering an effective amount of a vaccine or pharmaceutical composition comprising RSV G protein, viral particle/virus like particle, nucleic acid, attenuated RSV strain, or vector as reported herein to a subject in need thereof. In certain embodiments, the subject is pregnant woman, a premature baby, newborn, infant, child, adult, elderly, or immune compromised.

In certain embodiments, the subject is a human subject is 2, 12, or 16 years old or older. In certain embodiments, the subject is a human subject 2, 12, or 15 years old or less than 2, 12, or 16 years old. In certain embodiments, the subject is a human subject 55 or 65 years old or older. In certain embodiments, the subject is an infant, e.g., from one month to two years of age. In certain embodiments, the subject is a child, e.g., from one two to twelve years of age. In certain embodiments, the subject is an adolescent, e.g., from twelve to sixteen years of age. In certain embodiments, the subject is a human subject sixteen years of age or older.

In certain embodiments, the subject is immune compromised due to the need to maintain an immune suppressive drug(s) therapy, e.g., the subject is or diagnoses with DiGeorge syndrome, Wiskott-Aldrich syndrome, Bruton's agammaglobulinemia, the subject is receiving (being administered) chemotherapy or radiation due to being diagnosed with cancer, the subject is receiving corticosteroids due to a diagnosis of rheumatoid arthritis, lupus, vasculitis, or the subject is a solid organ recipient to prevent rejection of the transplanted organ.

In certain embodiments, it is contemplated that the compositions and methods of this disclosure provide certain advantages such as inducing higher levels of neutralizing antibodies, increasing neutralizing titers, an increased response to vaccination that is associated with a decreased likelihood of RSV-associated acute respiratory illness. Other contemplated advantages may be increased stability and shelf life of pharmaceutical compositions, avoiding the need to involve the use of chemicals (e.g., formalin) that could possibly modify protective epitopes, resulting in decreased immunogenicity and, possibly, preventing the induction of enhanced respiratory disease (ERD).

### Pharmaceutical Compositions

In certain embodiments, this disclosure relates to vaccine or pharmaceutical compositions comprising an RSV G protein, viral particle/virus like particle, virosome, nucleic acid, attenuated RSV stain, attenuated RSV virus, or vector as reported herein to a subject in need thereof. The pharmaceutical compositions provided herein may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

A vaccine is typically understood to be a prophylactic or therapeutic material providing at least one antigen or antigenic function. Typically, the vaccine is comprised of a conventional saline or buffered aqueous solution medium in which the composition is suspended or dissolved. Upon introduction into a host, the vaccine provokes an immune response including, but not limited to, the production of antibodies and/or cytokines and/or the activation of cytotoxic T cells, antigen presenting cells, helper T cells, dendritic cells and/or other cellular responses.

In certain embodiments, the vaccine optionally includes an adjuvant, i.e., a non-specific stimulator of the immune response or substances that allow generation of a depot in the host which when combined with the vaccine provides for an even more enhanced immune response. Examples include incomplete Freund's adjuvant and aluminum hydroxide.

In certain embodiments, the vaccine is a nucleic acid, recombinant vector, or attenuated vaccine, e.g., DNA, RNA, or mRNA-based vaccine which encodes a peptide antigen(s) disclosed herein having at least one open reading frame that can be translated by a cell or an organism provided with the nucleic acid, DNA, RNA, or mRNA. If more than one protein is translated, the proteins can be expressed in one vector/nucleic acid or in multiple (a plurality of) separate nucleic acids/vectors. The product of translation is a peptide or protein disclosed herein or viral particle containing such a protein or proteins, that may act as an antigen, preferably as an immunogen. The product may also be a fusion protein composed of more than one immunogen, e.g., a fusion protein that has two or more peptides or proteins derived from the same or different virus-proteins, wherein the peptides or proteins are optionally linked by self-cleaving linker sequences.

In certain embodiments, a nucleic acid, DNA, RNA, or mRNA may be designed to have two (bicistronic) or more (multicistronic) open reading frames (ORF). An open reading frame in this context is a sequence including a start codon that can be used as a location to start translation of the encoded nucleic acid into a peptide or protein. Translation of such nucleic acid(s) yields two (bicistronic) or more (multicistronic) distinct translation products/proteins (provided the ORFs are not identical). For expression in eukaryotes such nucleic acids may comprise an internal ribosomal entry site (IRES) sequence which allows for expression of two or more proteins on a single nucleic acid molecule.

In certain embodiments, the peptides, virus particles, virus-like particles, nucleic acid, recombinant vector, virosomes, or attenuated vaccine, may be administered naked without being associated with any further vehicle.

In certain embodiments, the peptides, virus particles, virus-like particles, nucleic acids, recombinant vectors, virosomes, or attenuated vaccines may be administered in a pharmaceutical composition having a pharmaceutically acceptable excipient selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiment, the pharmaceutically acceptable excipient is a cationic or polycationic compound and/or with a polymeric carrier. In certain embodiments, the peptides, virus particles, virus-like particles, virosomes, nucleic acids, recombinant vectors, or attenuated RSV are in a pharmaceutical composition associated with or complexed with a cationic or polycationic compound or a polymeric carrier and optionally an adjuvant. In certain embodiments, the cationic or polycationic compound is protamine, spermine, spermidine, poly-L-lysine (PLL), poly-histidine, or poly-arginine, cationic polysaccharides, such as chitosan, polybrene, cationic polymers, polyethyleneimine (PEI), homo- and co-polymers of lactic acid and glycolic acid, or polymethylmethacrylate.

In certain embodiments, the peptides, virus particles, virus-like particles, virosomes, nucleic acids, recombinant vectors, or attenuated RSV may be contained in a pharmaceutical composition with or administered in combination with an adjuvant. Contemplated adjuvants suitable for depot and delivery are cationic or polycationic compounds, liposomes, chitosan, alum solution, aluminium hydroxide, aluminium salts, aluminium phosphate gel, aluminium hydroxide gel (alum), polyphosphazene, squalene, squalene water emulsion, CpG oligonucleotides (nucleic acids with unmethylated CpG motifs), 1-alpha 25-dihydroxyvitamin D3, calcium phosphate gel, dimethyl dioctadecyl ammonium bromide, dehydroepiandrosterone, dimyristoylphosphatidylcholine, myristoyl phosphatidylglycerol, deoxycholic acid sodium salt, imiquimod, interferon gamma, interleukin-1 beta, interleukin-2, interleukin-7, interleukin-12, 7-allyl-8-oxoguanosine, acetylmuramyl-alanyl-isoglutamine, N-acetyl muramyl-L-threonyl-D-isoglutamine, NAc-Mur-L-Ala-D-Gln-OCH3, QS-21, Quil-A (Quil-A saponin), sorbitan trioleate, 2,6, 10,15,19,23-hexamethyltetracosan, stearyl tyrosine, lipid A, 4′-monophosphoryl lipid A, 3-O-desacyl-4′-monophosphoryl lipid A (MPLTM), liposomes containing lipid A, lipid A adsorbed on aluminium hydroxide.

Pharmaceutically acceptable carriers that may be used in these compositions include alumina, aluminum stearate, lecithin, serum proteins, albumin, such as human serum albumin, buffer substances such as phosphates, glycine, sorbic acid, citric acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, hydrophilic polymers such as polyvinyl pyrrolidone, cellulose based substances, polyethylene glycol, sodium carboxymethylcellulose, polyacrylates, waxes, gelatin, polyethylene polyoxypropylene block polymers, polyethylene glycol and antioxidants including ascorbic acid and methionine; preservatives; low molecular weight (less than about 10 residues) polypeptides; proteins; and amino acids such as glycine, glutamine, asparagine, histidine, arginine, or lysine. In certain embodiments, the excipient may be one or more selected from the list consisting of NaCl, trehalose, sucrose, mannitol, or glycine.

The disclosure also encompasses products obtainable by further processing of a liquid formulation, such as a frozen, lyophilized, or spray-dried product. Upon reconstitution, these solid products can become liquid formulations as described herein. In its broadest sense, therefore, the term “formulation” encompasses both liquid and solid formulations. However, solid formulations are understood as derivable from the liquid formulations (e.g., by freezing, freeze-drying, or spray-drying), and hence have various characteristics that are defined by the features specified for liquid formulations herein.

In certain embodiments, the formulations are isotonic in relation to human blood. Isotonic solutions possess the same or similar concentration of salts and water for maintaining osmotic balance with blood plasma so that they can be intravenously infused into a subject without substantially changing the osmotic pressure of the blood plasma.

In certain embodiments, it is contemplated that attenuated RSVs, recombinant vectors, and nucleic acids, virus particles, and peptides may be stored as a unit dosage form with a titer formulated in about 10 mM Tris, 140 mM NaCl pH 7.7. For the preparation of vaccine shots, e.g., 10²-10⁸ or 10²-10⁹ virus particles or virus like particles, can be lyophilized in phosphate-buffered saline (PBS) in the presence of peptone and human albumin in a sealed container, preferably a glass ampoule. Alternatively, the vaccine shots can be produced by stepwise freeze-drying a formulation. This formulation can contain additional additives such as mannitol, dextran, sugar, glycine, lactose or polyvinylpyrrolidone or other aids such as antioxidants or inert gas, stabilizers, or recombinant proteins (e.g., human serum albumin) suitable for in vivo administration. For vaccination or therapy, the lyophilisate can be dissolved in an aqueous solution, preferably physiological saline, or tris(hydroxymethyl)aminomethane buffer. In certain embodiments, the vaccine or pharmaceutical composition is administered intranasally, intradermally, or intramuscularly.

In certain embodiments, the vaccine or pharmaceutical composition is administered either systemically or locally, i.e., parenteral, subcutaneous, intravenous, intramuscular, intradermal, intranasal, aerosolized, or any other path of administration. The mode of administration, the dose and the number of administrations can be optimized.

In certain embodiments, this disclosure relates to kits containing materials useful for the vaccination, treatment, or prevention of an RSV infection. In certain embodiments, the kit comprises a container, a product label and a package insert. Suitable containers include, for example, bottles, vials, syringes, and boxes containing the same. The containers may be of a variety of materials, e.g., glass, plastic, or cardboard. The container holds the composition which is effective in treating the condition and may have a sterile access port (for example the container may be an intravenous solution bag or a vial having a stopper pierceable by a hypodermic injection needle). At least one active agent in the composition is a peptide, virus particle/virus like particle, nucleic acid, vector, or attenuated virus as disclosed herein. The product label on, or associated with, the container indicates that the composition is used for treating the condition of choice. In certain embodiments, the kit may further comprise a second container comprising a pharmaceutically acceptable buffer, such as a phosphate buffer saline or a citrate buffered saline. It may further include other materials desirable for a user or from commercial standpoint, including other buffers, diluents, filters, needles, and syringes. In certain embodiments, a dosage unit form can be, e.g., in the format of a prefilled syringe, an ampoule, cartridge or a vial.

In certain embodiments, this disclosure relates to kits or articles of manufacture, comprising a polypeptide, virus particle, nucleic acid, vector, or attenuated virus formulation thereof as disclosed herein and instructions for use by, e.g., a healthcare professional. The kits or articles of manufacture may include a container, vial, or a syringe containing the formulation as described herein.

Preferably, the container, vial, or syringe is composed of glass, plastic, or a polymeric material chosen from an olefin polymer or copolymer. The syringe, ampoule, cartridge, or vial can be manufactured of any suitable material, such as glass or plastic and may include rubber materials, such as rubber stoppers for vials and rubber plungers and rubber seals for syringes and cartridges. In certain embodiments, the kit may further comprise instructions for use and/or a clinical package leaflet. In certain embodiments, this disclosure also relates to packaging material, instructions for use, and/or clinical package leaflets, e.g., as required by regulatory aspects.

### RSV Live-Attenuated Vaccine Candidate Lacking G Protein Mucin Domains is Effective in Preventing RSV in BALB/c Mice

RSV live-attenuated vaccine (LAV) candidates were generated by removing the G-protein mucin domains to attenuate viral replication while retaining immunogenicity through deshielding of surface epitopes. Two LAV candidates were generated from recombinant RSV A2-line 19F by deletion of the G-protein mucin domains (A2-line19F-G155) or deletion of the G-protein mucin and a portion of the transmembrane domains (A2-line19F-G155S). Vaccine attenuation was measured in BALB/c mouse lungs by fluorescent focus unit (FFU) assays and real-time polymerase chain reaction (RT-PCR). Immunogenicity was determined by measuring serum binding and neutralizing antibodies in mice following prime/boost on days 28 and 59. Efficacy was determined by measuring RSV lung viral loads on day 4 post-challenge. Both LAVs were undetectable in mouse lungs by FFU assay and elicited similar neutralizing antibody titers compared to A2-line19F on days 28 and 59. Following RSV challenge, vaccinated mice showed no detectable RSV in the lungs by FFU assay and a significant reduction in RSV RNA in the lungs by RT-PCR of 560-fold for A2-line19F-G155 and 604-fold for A2-line19FG155S compared to RSV-challenged, unvaccinated mice. These experiments indicate that removal of the G-protein mucin domains produced RSV LAV candidates that were highly attenuated with retained immunogenicity.

RSV is an enveloped, negative-sense, single-stranded RNA virus, and its genome contains 10 genes encoding 11 known proteins. Among these, the surface glycoproteins F (which mediates viral fusion) and G (which facilitates attachment) are the predominant immunogens, capable of eliciting neutralizing antibodies in vivo. G is a heavily glycosylated with about 298-amino acids having 2 large, variable, mucin-like domains that flank a highly conserved CX3C motif within the central conserved domain (CCD). G exists in transmembrane bound and secreted forms, and the secreted form may function as an antigen decoy, interfering with antibody-mediated immune responses. Deletion of the entire G protein attenuates viral replication.

The glycosylated regions of some viral glycoproteins can function as steric shields, masking surface epitopes from recognition by the host immune system and facilitating immune evasion. Experiments were performed to determine whether removal of the heavily glycosylated mucin domains from RSV G would generate a highly attenuated vaccine candidate with impaired viral attachment but preserved immunogenicity due to deshielding of immunodominant epitopes.

### Assembly and Rescue of Recombinant RSV Viruses

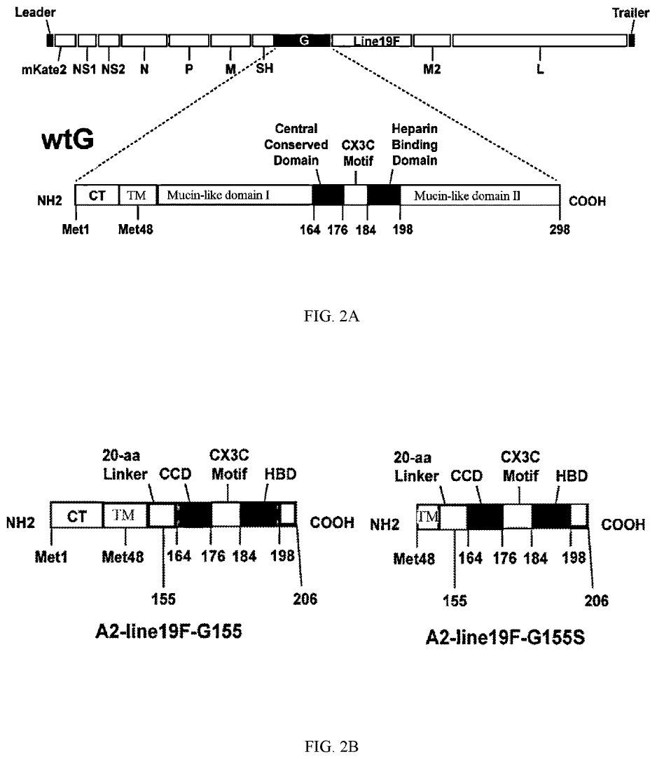

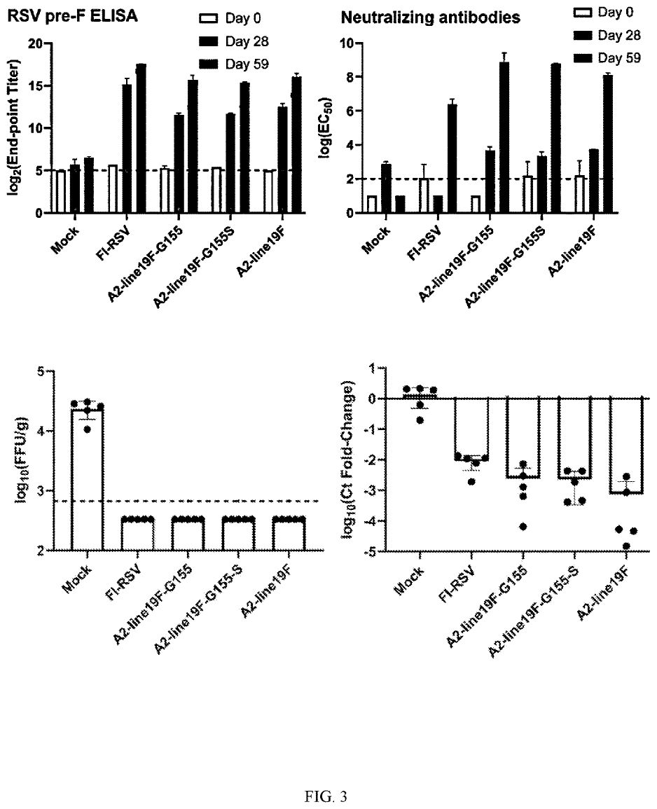

The rescue of recombinant A2-line19F, which expresses mKate2 and the RSV strain line19 fusion protein in an A2 backbone, was used. To generate recombinant viruses expressing modified G proteins within the A2-line19F backbone, synthetic G nucleotide sequences were obtained flanked by SacI-SacII restriction sites that were used to clone the corresponding G genes into the pSynkRSV-A2-line19F bacterial artificial chromosome. The resultant strain A2-line19F-G155 had deletion of the G-protein mucin domains, whereas strain A2-line19F-G155S had deletion of both the G protein mucin domains and a portion of the transmembrane domain such that it only expressed a secreted G protein lacking mucin (FIGS. 2A and 2B). To recover the recombinant viruses, BSR-T7/5 cells were cotransfected with RSV antigenomic bacterial artificial chromosomes and 4 human codon-optimized helper plasmids expressing either RSV N, P, M2-1, and L, as reported in Hotard et al. A stabilized respiratory syncytial virus reverse genetics system amenable to recombination-mediated mutagenesis. Master and working virus stocks were generated and harvested from Vero cells, flash-frozen in liquid nitrogen, and stored at negative 80° C. until use. RNA from viral stocks was extracted and the F and G genes were sequence confirmed by Sanger sequencing. Whole viral genome sequencing confirmed the entire sequence identify of A2-line19F-G155 and identified a single mutation in A2-line19F-G155S large polymerase protein gene that resulted in a K1766E amino acid change that did not lie within a functionally active or conserved domain.

### Cell Culture

HEp-2, Vero, and BSR-T7/5 cells were cultured. The recombinant viruses analyzed in this study express monomeric Katushka 2 (mKate2), a far-red fluorescent reporter protein located in the first gene position. Studies indicated recombinant expression of mKate2 did not cause viral attenuation. See Stobart et al., Nat Commun, 2016, 7:13916 and Hotard et al., Virology, 2012, 434:129-36.

Primary normal human bronchial epithelial (NHBE) cells were isolated from human donor lung explants and cultured at an air-liquid interface (ALI). Cells were expanded in co-culture with irradiated 3T3 cells in F+Y Reprogramming Medium and then plated on plates (0.4 μM pore size, polyester). After 2 days the cells were transitioned to ALI and differentiated in E-ALI medium. Once cultures were at ALI, the medium was changed every 48-72 h and cultures were allowed to differentiate for at least 3 weeks before experimentation.

### Assembly and Rescue of Recombinant RSV Viruses

To generate recombinant viruses expressing modified G proteins within the A2-line19F backbone, synthetic G nucleotide sequences were obtained, flanked by SacI-SacII restriction sites that were used to clone the corresponding G genes into the pSynkRSV-A2-line19F bacterial artificial chromosome (BAC). The resultant strain A2-line19F-G155 had deletion of the G-protein mucin domains, whereas strain A2-line19F-G155S had deletion of both the G protein mucin domains and the transmembrane domain such that it only expressed a secreted G protein lacking mucin. To recover the recombinant viruses, BSR-T7/5 cells were co-transfected with RSV antigenomic BACs and four human codon-optimized helper plasmids expressing either RSV N, P, M2-1, and L. Master and working virus stocks were generated and harvested from Vero cells, flash-frozen in liquid nitrogen, and stored at −80° C. until use. RNA from viral stocks was extracted and the F and G genes were sequence-confirmed by Sanger sequencing. Whole viral genome sequencing confirmed the entire sequence identify of A2-line19F-G155 and identified a single mutation in A2-line19F-G155S large polymerase protein gene that resulted in a K1766 E amino acid change that did not lie within a functionally active or conserved domain.

### Characterization of Protein Expression

To measure protein expression, Western blots were performed on infected HEp-2 lysates and supernatants. Monoclonal antibody binding of motavizumab (anti-F) and 3D3 (anti-G) to whole virus was also measured using an enzyme-linked immunosorbent assay (ELISA).

Surface expression of F and G on infected cells was measured by immunofluorescence using flow cytometry. HEp-2 cells were infected with A2-line19F or the vaccine strains and incubated at 37° C. overnight. The following day, cells were incubated with motavizumab and 3D3 monoclonal antibodies and subsequently stained with Allophycocyanin (APC)-conjugated goat anti-human IgG (H+L) antibody and Brilliant Violet 421 goat anti-mouse IgG for 30 min. Images were acquired on an LSR II flow cytometer and analyzed. Gating was performed on live cells and singlets, and infected cells were gated based on mKate2 expression.

### Viral Replication In Vitro

To measure viral growth kinetics in vitro, six-well plates containing Vero cells at 70-90% confluency were infected in duplicate at a multiplicity of infection (MOI) of 0.01 in 500 μL/well. At hours 6, 24, 48, 72, 96, and 120 post-infection, cell monolayers were scraped into supernatant, vortexed, flash-frozen in liquid nitrogen, and stored at −80° C. until titrating. NHBE cells differentiated at ALI were infected in triplicate at MOI of 8.0 based on a seeded cell density of 150,000 at 100 μL/insert. Cells were washed with Emory-ALI medium, and virus inoculum was applied apically for 2 h at 37° C. At hours 0 and 24, 48, 72, 96, and 120 post-infection, 150 μL full medium was applied apically twice for 10 min at 37° C. The 300 μL/insert was flash-frozen in liquid nitrogen and stored at −80° C. until titrating. Virus titers were determined using a fluorescent focus unit (FFU) quantification assay 48 hours post-infection on HEp-2 cells.

### Viral Replication of G-Mutant Vaccines in BALB/c Mice

To ascertain the level of viral attenuation in vivo, we infected groups of 5 BALB/c mice intranasally (IN) with 106 FFU of A2-line19F, A2-line19F-G155, or A2-line19F-G155S under ketamine/xylazine anesthesia. On day 4 post-infection, we euthanized the mice using pentobarbital, harvested the left lung, homogenized, and measured lung viral load using the FFU assay and real-time polymerase chain reaction (RT-PCR) (Supplemental Methods). The ΔCt (difference in cycle threshold) between the RSV matrix M (gene of interest) and GAPDH (endogenous control) genes were calculated for each sample and averaged for two replicates. The ΔΔCt (difference between each sample's ΔCt and the mean positive control (A2-line19F) ΔCt) was calculated to determine viral replication relative to A2-line19F. The linear fold-change in RSV M gene expression levels for a given sample relative to A2-line19F was calculated using the formula 2 (−ΔΔCt).

### Immunogenicity of G-Mutants in BALB/c Mice

To ascertain immunogenicity of the G-mutant vaccines in BALB/c mice, groups of 5 BALB/c mice IN were infected with 10⁶ FFU of A2-line19F, A2-line19F-G155, A2-line19F-G155S, or mock (plain MEM) with a prime/boost regimen on Days 1 and 29. Another group of mice had intramuscular administration of 50 μL of formalin-inactivated A2-line19F (FI-RSV) at the same time points. Serum was collected by submandibular bleeding on Days 0, 28, and 59. For the wellbeing of the animals, the blood collection was performed on the day preceding vaccination or challenge rather than the same day. Samples were centrifuged at 8000×g for 10 minutes, the supernatant removed, pooled for each group, heat-inactivated at 56° C. for 30 minutes, and stored at −80° C. until testing.

To measure immunogenicity, mouse sera were analyzed by ELISA for binding antibodies to RSV full-length A2-F, pre-fusion stabilized A2-F, and G central conserved domain (CCD) peptide; and by neutralization assays to A2-line19F. RSV F and G CCD were analyzed as single replicates due to limitations in sample volume, whereas RSV pre-fusion F was analyzed in two replicates and the GMT end-point titer determined. Neutralization assays were performed, and EC₅₀ values were generated, and the GMT of two replicates of each pooled specimen was determined.

### Vaccine Efficacy of G-Mutants in BALB-c Mice

To determine vaccine efficacy against RSV challenge, the same mice described above were then challenged with 10⁶ FFU IN of A2-line19F on Day 60. Four days after challenge, the mice were euthanized, and the left lungs were harvested and homogenized for viral titration by FFU assay and RT-PCR. RT-PCR was performed analogously to the mouse lung attenuation experiment detailed above, with exception that the ΔΔCt between each sample's ΔCt and the mean mock ΔCt was calculated to determine viral replication relative to mock.

To determine the potential for the vaccines to elicit enhanced histopathology after RSV challenge, the right lungs were also excised at the time of euthanasia and placed in 10% neutral buffered formalin. Samples were embedded in paraffin, cut 4-μm sections, and stained with hematoxylin and eosin (H&E). Histopathologic scores were performed by a blinded veterinary pathologist for three parameters eosinophil recruitment, perivascular cuffing, and interstitial pneumonia. A fourth parameter of pulmonary edema was added. Each parameter was scored separately for each histopathologic section, with a maximum value of 4 and a minimum of 0. Immunohistochemistry staining was also performed to ascertain the presence of RSV antigen in the lungs using goat polyclonal anti-RSV antibody.

### Growth Kinetics of G-Mutant Viruses

To assess the impact of the removal of mucin domains on viral growth kinetics in vitro, Vero cells and NHBE cells were infected and differentiated at ALI. Both A2-line19F-G155 and A2-line19F-G155S grew to similar titers as A2-line19F in Vero cells. However, in NHBE cells at ALI, which best approximate viral growth kinetics in seronegative infants, the vaccine candidates were significantly attenuated compared to both A2 and A2-line19F.

### Viral Growth Kinetics in BALB/c Mice

To measure the level of vaccine attenuation in vivo, infected groups of 5 BALB/c mice were challenged IN with 10⁶ FFU of either A2-line19F, A2-line19F-G155, or A2-line19F-G155S. On day 4 post-infection, mice were euthanized, lungs were harvested and homogenized, and viral lung titer was measured by FFU assay and RT-PCR. By the live-virus FFU assay, A2-line19F-G155 and A2-line19F-G155S were undetectable in the mouse lungs, as compared to A2-line19F, which had a geometric mean titer (GMT) of 4.62 log 10 FFU/g lung (fold-reduction >124-fold). By RT-PCR, which also has the potential to detect nonreplicating viral RNA, detection as measured by Ct fold-change was reduced 3-fold for A2-line19F-G155S and 22-fold for A2-line19F-G155 compared to A2-line 19F.

### Immunogenicity of G Mutants in BALB/c Mice

The immunogenicity of the vaccine candidates in BALB/c mice using a prime/boost regimen was measured on days 1 and 29, by vaccinating mice with A2-line19F-G155, A2-line19F-G155S, A2-line19F, mock, or FI-RSV (intramuscular) and challenging with RSV A2-line19F on day 60. Binding and neutralizing antibody responses were measured by RSV F ELISA, RSV G-CCD ELISA, RSV prefusion F ELISA, and neutralizing antibodies to A2-line19F in HEp-2 cells. A2-line19F-G155 and A2-line19F-G155S vaccines elicited similar levels of RSV F and prefusion F binding antibodies compared to A2-line19F on days 28 and 59. In contrast, binding antibodies to the G-CCD elicited by the vaccine candidates were numerically lower than those elicited by A2-line19F or FI-RSV. Both vaccine candidates elicited similar titers of neutralizing antibodies compared to A2-line19F on day 28. Neutralizing antibody titers significantly increased with the boost dose of vaccination, regardless of the priming vaccine.

### Vaccine Efficacy of G Mutants in BALB/c Mice

To assess for vaccine efficacy, these same mice were then challenged IN with 10⁶ FFU of A2-line19F on day 60 and the left lungs were harvested 4 days post challenge. Both G-mutant vaccines reduced lung viral titers to undetectable levels by FFU assay after RSV challenge, comparable to wild-type A2-line19F and FI-RSV. By RT-PCR, vaccination reduced lung viral RNA detection 560-fold for A2-line19F-G155, 604-fold for A2-line19F-G155S, and 1812-fold for A2-line19F when compared to mock-challenged mice.

What is claimed is:

1. A recombinant synthetic RSV G protein comprising the amino acid sequence of
(SEQ ID NO. 1)
AAIIFIASANHKVTPTTAIIQDATSQIKNTPPS.

2. The RSV G protein of claim 1 comprising the amino acid sequence of
(SEQ ID NO: 2)
MIISTSLIIAAIIFIASANHKVTPTTAIIQDATSQIKNTPPSKPN
NDFHFEVFNFVPCSICSNNPTCWAICKRIPNKKPGKKTTTKPTKK
P.

3. The RSV G protein of claim 1 comprising the amino acid sequence of
(SEQ ID NO: 3)
MSKNKDQRTAKTLERTWDTLNHLLFISSCLYKLNLKSVAQITLSI
LAMIISTSLIIAAIIFIASANHKVTPTTAIIQDATSQIKNTPPSK
PNNDFHFEVFNFVPCSICSNNPTCWAICKRIPNKKPGKKTTTKPT
KKP.

4. The RSV G protein of claim 1 wherein 50 or more amino acids in the C-terminal mucin like domain II are deleted.

5. The RSV G protein of claim 1 wherein 50 or more amino acids in the C-terminal mucin like domain I are deleted.

6. A nucleic acid encoding the recombinant RSV G protein as provided in claim 1.

7. The nucleic acid of claim 6 which is RNA or DNA in operable combination with a promoter.

8. A vector comprising a nucleic acid of claim 6.

9. A live attenuated RSV comprising an RSV G protein as provided in claim 1.

10. A host cell comprising a nucleic acid or vector encoding an RSV G protein as provided in claim 1.

11. An immunogenic composition comprising a nucleic acid encoding an RSV G protein as provided in claim 1 and a pharmaceutically acceptable excipient.

12. An immunogenic composition comprising an RSV G protein as provided in claim 1 and a pharmaceutically acceptable excipient.

13. An immunogenic composition comprising a particle having an RSV G protein as provided in claim 1 and a pharmaceutically acceptable excipient.

14. A method for producing an immunogenic composition against RSV comprising providing a host cell culture; inoculating the host cell culture with a nucleic acid encoding an RSV G protein as provided in claim 1; incubating the cell culture with the RSV; harvesting RSV following the incubation step; and formulating the harvested RSV into an immunogenic composition against RSV.

15. A method for inducing an effective immune response against RSV infection in a subject, comprising administering to the subject an immunologically effective dose of the immunogenic composition against RSV as in claim 11.

16. A method of vaccinating a subject for RSV comprising administering an effective amount of a composition as in claim 12.

17. The method of claim 15, wherein the subject is a human patient.

18. The method of claim 15, wherein the subject is a pregnant mother, newborn, or child less than 1 year old.

19. The method of claim 15, wherein the subject is a child less than 2 years old.

20. The method of claim 15, wherein the subject is 2 to 12 years old.
